(19) United States
(12) Patent Application Publication
(10) Pub. No.: US 2006/0229310 A1
(43) Pub. Date: Oct. 12, 2006

(54) Antibacterial compounds

(76) Inventor: Frank L. Wagenaar, Gurnee, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/301,502

(22) Filed: Dec. 13, 2005

Related U.S. Application Data
(60) Provisional application No. 60/636,003, filed on Dec. 14, 2004.

(51) Int. Cl. A61K 31/53; A61K 31/506; A61K 31/501; A61K 31/497; A61K 31/4436; A61K 31/4433; A61K 31/427; A61K 31/421; A61K 31/4178; A61K 31/416; A61K 31/381; C07D 409/02; C07D 405/02

(52) U.S. Cl. 514/241; 514/252.01; 514/256; 514/255.05; 514/336; 514/365; 514/374; 514/397; 514/406; 514/444; 514/461; 544/209; 544/238; 544/333; 544/405; 546/280.1; 546/283.4; 548/134; 548/143; 548/202; 548/206; 548/215; 548/311.1; 548/365.7; 549/59; 549/60

(57) ABSTRACT

Antibacterial compounds, processes for making them and intermediates used in the processes, compositions containing them, and methods for treatment of bacterial infections using them are disclosed.

This application claims priority to U.S. Provisional Application 60/636,003, filed Dec. 14, 2004, the specification of which is hereby incorporated by reference into this application.

### TECHNICAL FIELD

This invention is directed to antibacterial compounds, processes for making them and intermediates used in the processes, compositions containing them, and methods for treatment of bacterial infections using them.

### BACKGROUND OF THE INVENTION

Because the effectiveness of many drugs currently available for prophylaxis and treatment of bacterial infections is being compromised by the emergence of drug-resistant bacteria, the introduction of novel antibacterial compounds would be beneficial for their therapeutic value and their contribution to the antibacterial arts.

### SUMMARY OF THE INVENTION

One embodiment of this invention, therefore, pertains to compounds, and therapeutically acceptable salts, prodrugs and salts of prodrugs thereof, which are useful for treating bacterial infections, said compounds having formula (I) 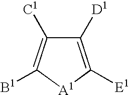
wherein

A¹ is O or S;

one of B¹ or C¹ is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;

D¹ is H, OH, OCH₃, F, Cl or Br; and

E¹ is CO₂H or tetrazolyl; or

A¹ is C(R²)═C(R³);

one of B¹, C¹ or R² is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;

R³ and D¹ are independently H, OH, OCH₃, F, Cl or Br;

E¹ is CO₂H or tetrazolyl; or

A¹ is C(H)═C(H);

D¹ and E¹ are together and, with the atoms to which each is attached, are furan-2-carboxylic acid or furan-2-tetrazolyl; and

one of B¹ or C¹ is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;

R¹ is C₁-alkyl, C₂-alkyl, C₃-alkyl, C₄-alkyl, C₅-alkyl, C₆-alkyl, C₃-alkenyl, C₄-alkenyl, C₅-alkenyl, C₆-alkenyl, C₃-alkynyl, C₄-alkynyl, C₅-alkynyl or C₆-alkynyl;

R⁴ is R⁵, R⁶, R⁷ or R⁸;

R⁵ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;

R⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two or three independently selected R⁹, OR⁹, SR⁹, S(O)R⁹, SO₂R⁹, NHR⁹, N(R⁹)₂, C(O)R⁹, C(O)NH₂, C(O)NHR⁹, NHC(O)R⁹, NHSO₂R⁹, NHC(O)OR⁹, SO₂NH₂, SO₂NHR⁹, SO₂N(R⁹)₂ or NHC(O)NHR⁹, substituents;

R⁹ is R¹⁰, R¹¹, R¹² or R¹³;

R¹⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R¹¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R¹² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;

R¹³ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two independently selected R¹⁴ substituents;

R¹⁴ is R¹⁵, R¹⁶, R¹⁷ or R¹⁸;

R¹⁵ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R¹⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R¹⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;

R¹⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two or three independently selected R¹⁹ substituents;

R¹⁹ is R²⁰, R²¹ or R²²;

R²⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R²¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R²² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;

wherein each foregoing cyclic moiety is independently unsubstituted, further unsubstituted, substituted or further substituted with one or two or three independently selected R²³, OR²³, SR²³, S(O)R²³, SO₂R²³, C(O)R²³, CO(O)R²³, OC(O)R²³, OC(O)OR²³, NH₂, NHR²³, N(R²³)₂, NHC(O)R²³, NR²³C(O)R²³, C(O)NH₂, C(O)NHR²³, C(O)N(R²³)₂, SO₂NH₂, SO₂NHR²³, SO₂N(R²³)₂, CF₃, CF₂CF₃, C(O)H, OH, NO₂, CF₃, CF₂CF₃, OCF₃, OCF₂CF₃, F, Cl, Br or I substituents;

R²³ is R²⁴, R²⁵, R²⁶ or R²⁷;

R²⁴ is phenyl which is unfused or fused with benzene;

R²⁵ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl;

R²⁶ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N; and

R²⁷ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two independently selected phenyl, furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl 1,2,3-triazolyl, CN, NH₂, NH(CH₃), N(CH₃)₂, CF₃, OCF₃, CF₂CF₃, OCF₂CF₃, F, Cl, Br or I substituents;

wherein the moieties represented by R²⁴, R²⁵ and R²⁶ are unsubstituted or substituted with one or two or three independently selected C₁-alkyl, C₂-alkyl, C₃-alkyl, C₄-alkyl, C₅-alkyl, C₆-alkyl, CN, NH₂, NH(CH₃), N(CH₃)₂, CF₃, OCF₃, CF₂CF₃, OCF₂CF₃, F, Cl, Br or I substituents.

Another embodiment pertains to compounds having formula (I), and therapeutically acceptable salts, prodrugs and salts of prodrugs thereof, wherein

A¹ is O or S;

one of B¹ or C¹ is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴. NR⁴C(O)R⁴, SO₂NHR⁴, SO₂N(R⁴)₂, NHSO₂R⁴ or NR⁴SO₂R⁴ substituents;

D¹ is H, OH, OCH₃, F, Cl or Br; and

E¹ is CO₂H or tetrazolyl; or

A¹ is C(R²)═C(R³);

one of B¹, C¹ or R² is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;

R³ and D¹ are independently H, OH, OCH₃, F, Cl or Br;

E¹ is CO₂H or tetrazolyl; or

A¹ is C(H)═C(H);

D¹ and E¹ are together and, with the atoms to which each is attached, are furan-2-carboxylic acid or furan-2-tetrazolyl; and

one of B¹ or C¹ is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;

R¹ is C₁-alkyl, C₂-alkyl, C₃-alkyl, C₄-alkyl, C₅-alkyl, C₆-alkyl, C₃-alkenyl, C₄-alkenyl, C₅-alkenyl, C₆-alkenyl, C₃-alkynyl, C₄-alkynyl, C₅-alkynyl or C₆-alkynyl;

R⁴ is R⁵, R⁶, R⁷ or R⁸;

R⁵ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene;

R⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;

R⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two or three independently selected R⁹, OR⁹, SR⁹, S(O)R⁹, SO₂R⁹, NHR⁹, N(R⁹)₂, C(O)R⁹, C(O)NH₂, C(O)NHR⁹, NHC(O)R⁹, NHSO₂R⁹, NHC(O)OR⁹, SO₂NH₂, SO₂NHR⁹, SO₂N(R⁹)₂ or NHC(O)NHR⁹, substituents;

R⁹ is R¹⁰, R¹¹, R¹² or R¹³;

R¹⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R¹¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene;

R¹² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;

R¹³ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two independently selected R¹⁴ substituents;

R¹⁴ is R¹⁵, R¹⁶, R¹⁷ or R¹⁸;

R¹⁵ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R¹⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene;

R¹⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;

R¹⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two or three independently selected R¹⁹ substituents;

R¹⁹ is R²⁰, R²¹ or R²²;

R²⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R²¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene;

R²² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;

wherein each foregoing cyclic moiety is independently unsubstituted, further unsubstituted, substituted or further substituted with one or two or three independently selected R²³, NH₂, NHR²³, NHC(O)R²³, OH, NO₂, CF₃, CF₂CF₃, OCF₃, OCF₂CF₃, F, Cl, Br or I substituents;

R²³ is R²⁴, R²⁵, R²⁶ or R²⁷;

R²⁴ is phenyl which is unfused or fused with benzene;

R²⁵ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl;

R²⁶ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH; and

R²⁷ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two independently selected phenyl, furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl 1,2,3-triazolyl, CN, NH₂, NH(CH₃), N(CH₃)₂, CF₃, OCF₃, CF₂CF₃, OCF₂CF₃, F, Cl, Br or I substituents;

wherein the moieties represented by R²⁴, R²⁵ and R²⁶ are unsubstituted or substituted with one or two or three independently selected C₁-alkyl, CN, NH₂, NH(CH₃), N(CH₃)₂, CF₃, OCF₃, CF₂CF₃, OCF₂CF₃, F, Cl, Br or I substituents.

Still another embodiment pertains to compounds having formula (I), and therapeutically acceptable salts, prodrugs and salts of prodrugs thereof, wherein

A¹ is O or S;

one of B¹ or C¹ is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;

D¹ is H, OH, OCH₃, F, Cl or Br; and

E¹ is CO₂H or tetrazolyl; or

A¹ is C(R²)═C(R³);

one of B¹, C¹ or R² is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;

R³ and D¹ are independently H, OH, OCH₃, F, Cl or Br;

E¹ is CO₂H or tetrazolyl; or

A¹ is C(H)═C(H);

D¹ and E¹ are together and, with the atoms to which each is attached, are furan-2-carboxylic acid or furan-2-tetrazolyl; and

one of B¹ or C¹ is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;

R¹ is C₁-alkyl, C₂-alkyl, C₃-alkyl, C₄-alkyl, C₅-alkyl, C₆-alkyl, C₃-alkenyl, C₄-alkenyl, C₅-alkenyl, C₆-alkenyl, C₃-alkynyl, C₄-alkynyl, C₅-alkynyl or C₆-alkynyl;

R⁴ is R⁵, R⁶, R⁷ or R⁸;

R⁵ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene;

R⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;

R⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two or three independently selected R⁹, OR⁹, SR⁹, S(O)R⁹, SO₂R⁹, NHR⁹, N(R⁹)₂, C(O)R⁹, C(O)NH₂, C(O)NHR⁹, NHC(O)R⁹, NHSO₂R⁹, NHC(O)OR⁹, SO₂NH₂, SO₂NHR⁹, SO₂N(R⁹)₂ or NHC(O)NHR⁹, substituents;

R⁹ is R¹⁰, R¹¹, R¹² or R¹³;

R¹⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R¹¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene;

R¹² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;

R¹³ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two independently selected R¹⁴ substituents;

R¹⁷ is R¹⁵, R¹⁶, R¹⁷ or R¹⁸;

R¹⁵ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R¹⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene;

R¹⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;

R¹⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two or three independently selected R¹⁹ substituents;

R¹⁹ is R²⁰, R²¹ or R²²;

R²⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R²¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene;

R²² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;

wherein each foregoing cyclic moiety is independently unsubstituted, further unsubstituted, substituted or further substituted with one or two or three independently selected R²³, NH₂, NHR²³, NHC(O)R²³, OH, NO₂, CF₃, CF₂CF₃, OCF₃, OCF₂CF₃, F, Cl, Br or I substituents;

R²³ is R²⁴, R²⁵, R²⁶ or R²⁷;

R²⁴ is phenyl which is unfused or fused with benzene;

R²⁵ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl;

R²⁶ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH; and

R²⁷ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two independently selected phenyl, furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl 1,2,3-triazolyl, CN, NH₂, NH(CH₃), N(CH₃)₂, CF₃, OCF₃, CF₂CF₃, OCF₂CF₃, F, Cl, Br or I substituents;

wherein the moieties represented by R²⁴, R²⁵ and R²⁶ are unsubstituted or substituted with one or two or three independently selected C₁-alkyl, CN, NH₂, NH(CH₃), N(CH₃)₂, CF₃, OCF₃, CF₂CF₃, OCF₂CF₃, F, Cl, Br or I substituents.

Still another embodiment pertains to compounds having formula (I), and therapeutically acceptable salts, prodrugs and salts of prodrugs thereof, wherein

A¹ is O or S;

one of B¹ or C¹ is H, OH, OR¹, CN, F, Cl or Br, and the other is phenyl or C₆-cycloalkyl, each of which is substituted with R⁴, OR⁴, SR⁴, S(O)R⁴, SO₂R⁴, C(O)NHR⁴, C(O)N(R⁴)₂, NHC(O)R⁴, SO₂NHR⁴ or NHSO₂R⁴;

D¹ is H; and

E¹ is CO₂H; or

A¹ is C(R²)═C(R³);

one of B¹, C¹ or R² is H, OH, OR¹, CN, F, Cl or Br, and the other is phenyl or C₆-cycloalkyl, each of which is substituted with R⁴, OR⁴, SR⁴, S(O)R⁴, SO₂R⁴, C(O)NHR⁴, NHC(O)R⁴, SO₂NHR⁴ or NHSO₂R⁴;

R³ and D¹ are independently H, OH, OCH₃, F, Cl or Br;

E¹ is CO₂H or tetrazolyl; or

A¹ is C(H)═C(H);

D¹ and E¹ are together and, with the atoms to which each is attached, are furan-2-carboxylic acid; and

one of B¹ or C¹ is H, OH, OR¹, CN, F, Cl or Br, and the other is phenyl or C₆-cycloalkyl, each of which is substituted with R⁴, OR⁴, SR⁴, S(O)R⁴, SO₂R⁴, C(O)NHR⁴, NHC(O)R⁴, SO₂NHR⁴ or NHSO₂R⁴;

R¹ is C₁-alkyl, C₂-alkyl, C₃-alkyl, C₄-alkyl, C₅-alkyl or C₆-alkyl;

R⁴ is R⁵, R⁶, R⁷ or R⁸;

R⁵ is phenyl which is unfused or fused with benzene;

R⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl;

R⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;

R⁸ is C₁-alkyl, C₂-alkyl, C₃-alkyl, C₄-alkyl, C₅-alkyl or C₆-alkyl, each of which is substituted with one or two or three independently selected R⁹, OR⁹, SR⁹, S(O)R⁹, SO₂R⁹, NHR⁹, C(O)R⁹, C(O)NHR⁹, NHC(O)R⁹, NHSO₂R⁹, NHC(O)OR⁹, SO₂NH₂, SO₂NHR⁹ or NHC(O)NHR⁹ substituents;

R⁹ is R¹⁰, R¹¹, R¹² or R¹³;

R¹⁰ is phenyl which is unfused or fused with benzene;

R¹¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl;

R¹² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;

R¹³ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two independently selected R¹⁴ substituents;

R¹⁴ is R¹⁵, R¹⁶, R¹⁷ or R¹⁸;

R¹⁵ is phenyl which is unfused or fused with benzene;

R¹⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl;

R¹⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;

R¹⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two or three independently selected R¹⁹ substituents;

R¹⁹ is R²⁰, R²¹ or R²²;

R²⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R²¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl;

R²² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;

wherein the phenyl and C₆-cycloalkyl moieties represented by B¹, C¹ or R² are further unsubstituted or substituted with R²³, NH₂, NHC(O)R²³, OH, NO₂ or Cl, wherein R²³ is morpholinyl or phenyl substituted with C₁-alkyl; and

the moieties represented by R¹⁵, R¹⁶ and R¹⁷ are unsubstituted or substituted with one or two independently selected C₁-alkyl, phenyl or OH substituents.

Still another embodiment pertains to compounds having formula (I) which are
3-chloro-4′-((cyclohex-3-yl-1-(1,3-thiazol-5-yl)propoxy)methyl)-6-fluoro-2′-methyl(1,1′-biphenyl)-4-carboxylic acid,
5-(4-((cyclohex-3-yl-1-(1,3-thiazol-5-yl)propoxy)methyl)-2-methylphenyl)-2-thiophenecarboxylic acid,
3′-chloro-4′-((cyclohex-3-yl-1-(1,3-thiazol-5-yl)propoxy)methyl)-4-methoxy-6′-methyl(1,1′-biphenyl)-3-carboxylic acid,
5-(4-((cyclohex-3-ylpropoxy)methyl)-2-methylphenyl)-2-thiophenecarboxylic acid,
2′-(2-methylphenyl)-4′-((cyclohex-3-yl-1-(1,3-thiazol-5-yl)propoxy)methyl)(1,1′-biphenyl)-4-carboxylic acid,
2′-(2-methylphenyl)-4′-((cyclohex-3-yl-1-(1,3-thiazol-5-yl)propoxy)methyl)(1,1′-biphenyl)-3-carboxylic acid,
2′-(2-methylphenyl)-4′-(((3-methylphenylmethyl)thio)methyl)(1,1′-biphenyl)-3-carboxylic acid,
4′-((cyclohex-3-yl-1-(1,3-thiazol-5-yl)propoxy)methyl)-2′-methyl(1,1′-biphenyl)-3-carboxylic acid,
4-(4-propylcyclohexyl)benzoic acid,
4′-((cyclohex-3-yl-2-(1,3-thiazol-5-yl)propoxy)methyl)(1,1′-biphenyl)-3-carboxylic acid,
2′-methyl-4′-(((3-methylphenylmethyl)thio)methyl)(1,1′-biphenyl)-4-carboxylic acid,
2′-chloro-4′-(((3-methylphenylmethyl)thio)methyl)(1,1′-biphenyl)-4-carboxylic acid,
2′-methyl-4′-((pentyloxy)methyl)(1,1′-biphenyl)-4-carboxylic acid,
4′-((3-cyclohexyl-1-(furan-2-yl)propoxy)methyl)-2′-methyl(1,1′-biphenyl)-4-carboxylic acid,
4′-((cyclohex-3-yl-1-(thien-2-yl)propoxy)methyl)-2′-methyl(1,1′-biphenyl)-4-carboxylic acid,
3′-chloro-4′-((cyclohex-3-yl-1-(1,3-thiazol-5-yl)propoxy)methyl)(1,1′-biphenyl)-4-carboxylic acid,
4′-((cyclohex-3-l-1-(1,3-thiazol-5-yl)propoxy)methyl)-2,2′-dimethyl(1,1′-biphenyl)-4-carboxylic acid,
5-(4-((cyclohex-3-ylpropoxy)methyl)-2-methylphenyl)-1-benzofuran-2-carboxylic acid,
4′-((cyclohex-3-yl-1-(1,3-thiazol-5-yl)propoxy)methyl)-3-fluoro-2′-methyl(1,1′-biphenyl)-4-carboxylic acid,
4′-((cyclohex-3-yl-1-(1,3-thiazol-5-yl)propoxy)methyl)-2-methoxy-2′-methyl(1,1′-biphenyl)-4-carboxylic acid,
5-(4′-((cyclohex-3-yl-1-(1,3-thiazol-5-yl)propoxy)methyl)-2′-methyl(1,1′-biphenyl)-4-yl)-1H-tetraazole,
4′-((cyclohex-3-yl-1-(1,3-thiazol-5-yl)propoxy)methyl)-3-hydroxy(1,1′-biphenyl)-4-carboxylic acid,
4-(4-butylcyclohexyl)benzoic acid,
2′-chloro-4′-((cyclohex-3-yl-1-(1,3-thiazol-5-yl)propoxy)methyl)(1,1′-biphenyl)-3-carboxylic acid,
4′-((cyclohex-3-ylpropoxy)methyl)-2′-methyl(1,1′-biphenyl)-3-carboxylic acid,
6-(4-((cyclohex-3-yl-1-(1,3-thiazol-5-yl)propoxy)methyl)-2-methylphenyl)-1-benzofuran-2-carboxylic acid,
5-(4′-((cyclohex-3-yl-1-(1,3-thiazol-5-yl)propoxy)methyl)-4-fluoro-2′-methyl(1,1′-biphenyl)-3-yl)-1H-tetraazole,
5-(4′-((cyclohex-3-yl-1-(1,3-thiazol-5-yl)propoxy)methyl)-3-fluoro-2′-methyl(1,1′-biphenyl)-4-yl)-1H-tetraazole,
4′-((cyclohex-3-yl-1-(1,3-thiazol-5-yl)propoxy)methyl)-2′-methyl(1,1′-biphenyl)-4-carboxylic acid,
4′-((cyclohex-3-yl-1-(1,3-thiazol-5-yl)propoxy)methyl)-4-hydroxy-2′-methyl(1,1′-biphenyl)-3-carboxylic acid,
5-(4′-((cyclohex-3-ylpropoxy)methyl)-2′-nitro(1,1′-biphenyl)-3-yl)-2H-tetraazole,
2′-amino-4′-((cyclohex-3-yl-1-(1,3-thiazol-5-yl)propoxy)methyl)(1,1′-biphenyl)-3-carboxylic acid,
4′-((cyclohex-3-yl-1-(1,3-thiazol-5-yl)propoxy)methyl)-2′-((2-morpholinylcarbonyl)amino)(1,1′-biphenyl)-3-carboxylic acid,
2-fluoro-4-(4-pentylcyclohexyl)benzoic acid,
2′-(3-aminopropyl)-4′-((cyclohex-3-yl-1-(1,3-thiazol-5-yl)propoxy)methyl)-3-fluoro(1,1′-biphenyl)-4-carboxylic acid,
3-fluoro-2′-methyl-4′-(((1-pentylpropyn-2-yl)oxy)methyl)(1,1′-biphenyl)-4-carboxylic acid,
4′-((cyclohex-3-yl-1-(naphth-2-yl)propoxy)methyl)-3-fluoro-2′-methyl(1,1′-biphenyl)-4-carboxylic acid,
3-fluoro-2′-methyl-4′-(((1-pentylpropyn-2-yl)oxy)methyl)(1,1′-biphenyl)-4-carboxylic acid,
3-fluoro-4′-(((isoprop-1-yl-3-phenylpropyn-2-yl)oxy)methyl)-2′-methyl(1,1′-biphenyl)-4-carboxylic acid,
3-fluoro-2′-methyl-4′-(((pent-1-yl-3-(thien-2-l)propyn-2-yl)oxy)methyl)(1,1′-biphenyl)-4-carboxylic acid,
3-fluoro-2′-methyl-4′-((1-methyl-2,2-diphenylethoxy)methyl)(1,1′-biphenyl)-4-carboxylic acid,
3-fluoro-4′-((1-(5-hydroxy-2-phenyl-1,3-oxazol-4-yl)ethoxy)methyl)-2′-methyl(1,1′-biphenyl)-4-carboxylic acid,
4′-((1,3-diphenylpropoxy)methyl)-3-fluoro-2′-methyl(1,1′-biphenyl)-4-carboxylic acid,
4′-((cyclohex-3-yl-1-(thien-2-yl)propoxy)methyl)-3-fluoro-2′-methyl(1,1′-biphenyl)-4-carboxylic acid,
4′-((1-(1,1′-biphenyl)-4-ylcyclohex-3-yl propoxy)methyl)-3-fluoro-2′-methyl(1,1′-biphenyl)-4-carboxylic acid,
3-fluoro-2′-methyl-4′-((3-phenyl-1-(thien-2-yl)propoxy)methyl)(1,1′-biphenyl)-4-carboxylic acid,
4′-((cyclohex-3-yl-1-(1,3-thiazol-2-yl)propoxy)methyl)-3-fluoro-2′-methyl(1,1′-biphenyl)-4-carboxylic acid,
4′-((cyclohex-3-yl-1-phenylpropoxy)methyl)-3-fluoro-2′-methyl(1,1′-biphenyl)-4-carboxylic acid and
4′-((cyclohex-3-yl-1-(4-methylphenyl)propoxy)methyl)-3-fluoro-2′-methyl(1,1′-biphenyl)-4-carboxylic acid, and therapeutically acceptable salts, prodrugs, and salts of prodrugs thereof.

Still another embodiment pertains to compositions for treating bacterial infection in a fish or a mammal, said compositions comprising an excipient and a compound having formula (I), or therepeutically acceptable salt, prodrug or salt of a prodrug thereof.

Still another embodiment pertains to methods of treating bacterial infection in a fish or a mammal, said methods comprising administering to the fish or the mammal a compound having formula (I), 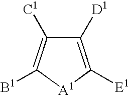
wherein

A¹ is O or S;

one of B¹ or C¹ is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;

D¹ is H, OH, OCH₃, F, Cl or Br; and

E¹ is CO₂H or tetrazolyl; or

A¹ is C(R²)═C(R³);

one of B¹, C¹ or R² is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;

R³ and D¹ are independently H, OH, OCH₃, F, Cl or Br;

E¹ is CO₂H or tetrazolyl; or

A¹ is C(H)═C(H);

D¹ and E¹ are together and, with the atoms to which each is attached, are furan-2-carboxylic acid or furan-2-tetrazolyl; and

one of B¹ or C¹ is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;

R¹ is C₁-alkyl, C₂-alkyl, C₃-alkyl, C₄-alkyl, C₅-alkyl, C₆-alkyl, C₃-alkenyl, C₄-alkenyl, C₅-alkenyl, C₆-alkenyl, C₃-alkynyl, C₄-alkynyl, C₅-alkynyl or C₆-alkynyl;

R⁴ is R⁵, R⁶, R⁷ or R⁸;

R⁵ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;

R⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two or three independently selected R⁹, OR⁹, SR⁹, S(O)R⁹, SO₂R⁹, NHR⁹, N(R⁹)₂, C(O)R⁹, C(O)NH₂, C(O)NHR⁹, NHC(O)R⁹, NHSO₂R⁹, NHC(O)OR⁹, SO₂NH₂, SO₂NHR⁹, SO₂N(R⁹)₂ or NHC(O)NHR⁹, substituents;

R⁹ is R¹⁰, R¹¹, R¹² or R¹³;

R¹⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R¹¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R¹² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;

R¹³ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two independently selected R¹⁴ substituents;

R¹⁴ is R¹⁵, R¹⁶, R¹⁷ or R¹⁸;

R¹⁵ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R¹⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R¹⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;

R¹⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two or three independently selected R¹⁹ substituents;

R¹⁹ is R²⁰, R²¹ or R²²;

R²⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R²¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R²² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;

wherein each foregoing cyclic moiety is independently unsubstituted, further unsubstituted, substituted or further substituted with one or two or three independently selected R²³, OR²³, SR²³, S(O)R²³, SO₂R²³, C(O)R²³, CO(O)R²³, OC(O)R²³, OC(O)OR²³, NH₂, NHR²³, N(R²³)₂, NHC(O)R²³, NR²³C(O)R²³, C(O)NH₂, C(O)NHR²³, C(O)N(R²³)₂, SO₂NH₂, SO₂NHR²³, SO₂N(R²³)₂, CF₃, CF₂CF₃, C(O)H, OH, NO₂, CF₃, CF₂CF₃, OCF₃, OCF₂CF₃, F, Cl, Br or I substituents;

R²³ is R²⁴, R²⁵, R²⁶ or R²⁷;

R²⁴ is phenyl which is unfused or fused with benzene;

R²⁵ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl;

R²⁶ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N; and

R² is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two independently selected phenyl, furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl 1,2,3-triazolyl, CN, NH₂, NH(CH₃), N(CH₃)₂, CF₃, OCF₃, CF₂CF₃, OCF₂CF₃, F, Cl, Br or I substituents;

wherein the moieties represented by R²⁴, R²⁵ and R²⁶ are unsubstituted or substituted with one or two or three independently selected C₁-alkyl, C₂-alkyl, C₃-alkyl, C₄-alkyl, C₅-alkyl, C₆-alkyl, CN, NH₂, NH(CH₃), N(CH₃)₂, CF₃, OCF₃, CF₂CF₃, OCF₂CF₃, F, Cl, Br or I substituents, or a therapeutically acceptable salt, prodrug or salt of a prodrug thereof.

### DETAILED DESCRIPTION OF THE INVENTION

Variable moieties herein are represented by identifiers (capital letters with numerical and/or alphabetical superscripts) and may be specifically embodied.

It is meant to be understood that proper valences are maintained for all moieties and combinations thereof, that monovalent moieties having more than one atom are drawn from left to right and are attached through their left ends, and that divalent moieties are also drawn from left to right.

It is also meant to be understood that a specific embodiment of a variable moiety herein may be the same or different as another specific embodiment having the same identifier.

The term “cyclic moiety,” as used herein, means arene, aryl, cycloalkane, cycloalkyl, cycloalkene, cycloalkenyl, heteroarene, heteroaryl, heterocycloalkane, heterocycloalkyl, heterocycloalkene and heterocycloalkenyl.

The term “arene,” as used herein, means benzene.

The term “aryl,” as used herein, means phenyl.

The term “cycloalkane,” as used herein, means C₃-cycloalkane, C₄-cycloalkane, C₅-cycloalkane and C₆-cycloalkane.

The term “cycloalkyl,” as used herein, means C₃-cycloalkyl, C₄-cycloalkyl, C₅-cycloalkyl and C₆-cycloalkyl.

The term “cycloalkene,” as used herein, means C₄-cycloalkene, C₅-cycloalkene and C₆-cycloalkene.

The term “cycloalkenyl,” as used herein, means C₃-cycloalkenyl, C₄-cycloalkenyl, C₅-cycloalkenyl and C₆-cycloalkenyl.

The term “heteroarene,” as used herein, means furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine and 1,2,3-triazole.

The term “heteroaryl,” as used herein, means furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thiophenyl, triazinyl and 1,2,3-triazolyl.

The term “heterocycloalkane,” as used herein, means cycloalkane having one or two or three CH₂ moieties replaced with independently selected O, C(O), CNOH, CNOCH₃, S, S(O), SO₂ or NH and one or two CH moieties unreplaced or replaced with N and also means cycloalkane having one or two or three CH₂ moieties unreplaced or replaced with independently selected O, C(O), CNOH, CNOCH₃, S, S(O), SO₂ or NH and one or two CH moieties replaced with N.

The term “heterocycloalkyl,” as used herein, means cycloalkyl having one or two or three CH₂ moieties replaced with independently selected O, C(O), CNOH, CNOCH₃, S, S(O), SO₂ or NH and one or two CH moieties unreplaced or replaced with N and also means cycloalkyl having one or two or three CH₂ moieties unreplaced or replaced with independently selected O, C(O), CNOH, CNOCH₃, S, S(O), SO₂ or NH and one or two CH moieties replaced with N.

The term “heterocycloalkene,” as used herein, means cycloalkene having one or two or three CH₂ moieties replaced with independently selected O, C(O), CNOH, CNOCH₃, S, S(O), SO₂ or NH and one or two CH moieties unreplaced or replaced with N and also means cycloalkene having one or two or three CH₂ moieties unreplaced or replaced with independently selected O, C(O), CNOH, CNOCH₃, S, S(O), SO₂ or NH and one or two CH moieties replaced with N.

The term “heterocycloalkenyl,” as used herein, means cycloalkenyl having one or two or three CH₂ moieties replaced with independently selected O, C(O), CNOH, CNOCH₃, S, S(O), SO₂ or NH and one or two CH moieties unreplaced or replaced with N and also means cycloalkenyl having one or two or three CH₂ moieties unreplaced or replaced with independently selected O, C(O), CNOH, CNOCH₃, S, S(O), SO₂ or NH and one or two CH moieties replaced with N.

The term “C₂-alkenyl,” as used herein, means ethenyl (vinyl).

The term “C₃-alkenyl,” as used herein, means 1-propen-1-yl, 1-propen-2-yl (isopropenyl) and 1-propen-3-yl (allyl).

The term “C₄-alkenyl,” as used herein, means 1-buten-1-yl, 1-buten-2-yl, 1,3-butadien-1-yl, 1,3-butadien-2-yl, 2-buten-1-yl, 2-buten-2-yl, 3-buten-1-yl, 3-buten-2-yl, 2-methyl-1-propen-1-yl and 2-methyl-2-propen-1-yl.

The term “C₅-alkenyl,” as used herein, means 2-methylene-3-buten-1-yl, 2-methylenebut-1-yl, 2-methyl-1-buten-1-yl, 2-methyl-1,3-butadien-1-yl, 2-methyl-2-buten-1-yl, 2-methyl-3-buten-1-yl, 2-methyl-3-buten-2-yl, 3-methyl-1-buten-1-yl, 3-methyl-1-buten-2-yl, 3-methyl-1,3-butadien-1-yl, 3-methyl-1,3-butadien-2-yl, 3-methyl-2-buten-1-yl, 3-methyl-2-buten-2-yl, 3-methyl-3-buten-1-yl, 3-methyl-3-buten-2-yl, 1-penten-1-yl, 1-penten-2-yl, 1-penten-3-yl, 1,3-pentadien-1-yl, 1,3-penta-dien-2-yl, 1,3-pentadien-3-yl, 1,4-pentadien-1-yl, 1,4-pentadien-2-yl, 1,4-pentadien-3-yl, 2-penten-1-yl, 2-penten-2-yl, 2-penten-3-yl, 2,4-pentadien-1-yl, 2,4-pentadien-2-yl, 3-penten-1-yl, 3-penten-2-yl, 4-penten-1-yl and 4-penten-2-yl.

The term “C₆-alkenyl,” as used herein, means 2,2-dimethyl-3-buten-1-yl, 2,3-dimethyl-1-buten-1-yl, 2,3-dimethyl-1,3-butadien-1-yl, 2,3-dimethyl-2-buten-1-yl, 2,3-dimethyl-3-buten-1-yl, 2,3-dimethyl-3-buten-2-yl, 3,3-dimethyl-1-buten-1-yl, 3,3-dimethyl-1-buten-2-yl, 2-ethenyl-1,3-butadien-1-yl, 2-ethenyl-2-buten-1-yl, 2-ethyl-1-buten-1-yl, 2-ethyl-1,3-butadien-1-yl, 2-ethyl-2-buten-1-yl, 2-ethyl-3-buten-1-yl, 1-hexen-1-yl, 1-hexen-2-yl, 1-hexen-3-yl, 1,3-hexadien-1-yl, 1,3-hexadien-2-yl, 1,3-hexadien-3-yl, 1,3,5-hexatrien-1-yl, 1,3,5-hexatrien-2-yl, 1,3,5-hexatrien-3-yl, 1,4-hexadien-1-yl, 1,4-hexadien-2-yl, 1,4-hexadien-3-yl, 1,5-hexadien-1-yl, 1,5-hexadien-2-yl, 1,5-hexadien-3-yl, 2-hexen-1-yl, 2-hexen-2-yl, 2-hexen-3-yl, 2,4-hexadien-1-yl, 2,4-hexadien-2-yl, 2,4-hexadien-3-yl, 2,5-hexadien-1-yl, 2,5-hexadien-2-yl, 2,5-hexadien-3-yl, 3-hexen-1-yl, 3-hexen-2-yl, 3-hexen-3-yl, 3,5-hexadien-1-yl, 3,5-hexadien-2-yl, 3,5-hexadien-3-yl, 4-hexen-1-yl, 4-hexen-2-yl, 4-hexen-3-yl, 5-hexen-1-yl, 5-hexen-2-yl, 5-hexen-3-yl, 2-methylene-3-methyl-3-buten-1-yl, 2-methylene-3-methylbut-1-yl, 2-methylene-3-penten-1-yl, 2-methylene-4-penten-1-yl, 2-methylenepent-1-yl, 2-methylenepent-3-yl, 3-methylene-1-penten-1-yl, 3-methylene-1-penten-2-yl, 3-methylenepent-1-yl, 3-methylene-1,4-pentadien-1-yl, 3-methylene-1,4-pentadien-2-yl, 3-methylene-pent-2-yl, 2-methyl-1-penten-1-yl, 2-methyl-1-penten-3-yl, 2-methyl-1,3-pentadien-1-yl, 2-methyl-1,3-pentadien-3-yl, 2-methyl-1,4-pentadien-1-yl, 2-methyl-1,4-pentadien-3-yl, 2-methyl-2-penten-1-yl, 2-methyl-2-penten-3-yl, 2-methyl-2,4-pentadien-1-yl, 2-methyl-2,4-pentadien-3-yl, 2-methyl-3-penten-1-yl, 2-methyl-3-penten-2-yl, 2-methyl-3-penten-3-yl, 2-methyl-4-penten-1-yl, 2-methyl-4-penten-2-yl, 2-methyl-4-penten-3-yl, 3-methyl-1-penten-1-yl, 3-methyl-1-penten-2-yl, 3-methyl-1,3-pentadien-1-yl, 3-methyl-1,3-pentadien-2-yl, 3-methyl-1,4-pentadien-1-yl, 3-methyl-1,4-pentadien-2-yl, 3-methyl-2-penten-1-yl, 3-methyl-2-penten-2-yl, 3-methyl-2,4-pentadien-1-yl, 3-methyl-3-penten-1-yl, 3-methyl-3-penten-2-yl, 3-methyl-4-penten-1-yl, 3-methyl-4-penten-2-yl, 3-methyl-4-penten-3-yl, 4-methyl-1-penten-1-yl, 4-methyl-1-penten-2-yl, 4-methyl-1-penten-3-yl, 4-methyl-1,3-pentadien-1-yl, 4-methyl-1,3-pentadien-2-yl, 4-methyl-1,3-pentadien-3-yl, 4-methyl-1,4-pentadien-1-yl, 4-methyl-1,4-pentadien-2-yl, 4-methyl-1,4-pentadien-3-yl, 4-methylene-2-penten-3-yl, 4-methyl-2-penten-1-yl, 4-methyl-2-penten-2-yl, 4-methyl-2-penten-3-yl, 4-methyl-2,4-pentadien-1-yl, 4-methyl-2,4-pentadien-2-yl, 4-methyl-3-penten-1-yl, 4-methyl-3-penten-2-yl, 4-methyl-3-penten-3-yl, 4-methyl-4-penten-1-yl and 4-methyl-4-penten-2-yl.

The term “C₁-alkyl,” as used herein, means methyl.

The term “C₂-alkyl,” as used herein, means ethyl.

The term “C₃-alkyl,” as used herein, means prop-1-yl and prop-2-yl (isopropyl).

The term “C₄-alkyl,” as used herein, means but-1-yl, but-2-yl, 2-methylprop-1-yl and 2-methylprop-2-yl (tert-butyl).

The term “C₅-alkyl,” as used herein, means 2,2-dimethylprop-1-yl (neo-pentyl), 2-methylbut-1-yl, 2-methylbut-2-yl, 3-methylbut-1-yl, 3-methylbut-2-yl, pent-1-yl, pent-2-yl and pent-3-yl.

The term “C₆-alkyl,” as used herein, means 2,2-dimethylbut-1-yl, 2,3-dimethylbut-1-yl, 2,3-dimethylbut-2-yl, 3,3-dimethylbut-1-yl, 3,3-dimethylbut-2-yl, 2-ethylbut-1-yl, hex-1-yl, hex-2-yl, hex-3-yl, 2-methylpent-1-yl, 2-methylpent-2-yl, 2-methylpent-3-yl, 3-methylpent-1-yl, 3-methylpent-2-yl, 3-methylpent-3-yl, 4-methylpent-1-yl and 4-methylpent-2-yl.

The term “C₂-alkynyl,” as used herein, means ethynyl (acetylenyl).

The term “C₃-alkynyl,” as used herein, means 1-propyn-1-yl and 2-propyn-1-yl (propargyl).

The term “C₄-alkynyl,” as used herein, means 1-butyn-1-yl, 1,3-butadiyn-1-yl, 2-butyn-1-yl, 3-butyn-1-yl and 3-butyn-2-yl.

The term “C₅-alkynyl,” as used herein, means 2-methyl-3-butyn-1-yl, 2-methyl-3-butyn-2-yl, 3-methyl-1-butyn-1-yl, 1,3-pentadiyn-1-yl, 1,4-pentadiyn-1-yl, 1,4-pentadiyn-3-yl, 2,4-pentadiyn-1-yl, 1-pentyn-1-yl, 1-pentyn-3-yl, 2-pentyn-1-yl, 3-pentyn-1-yl, 3-pentyn-2-yl, 4-pentyn-1-yl and 4-pentyn-2-yl.

The term “C₆-alkynyl,” as used herein, means 2,2-dimethyl-3-butyn-1-yl, 3,3-dimethyl-1-butyn-1-yl, 2-ethyl-3-butyn-1-yl, 2-ethynyl-3-butyn-1-yl, 1-hexyn-1-yl, 1-hexyn-3-yl, 1,3-hexadiyn-1-yl, 1,3,5-hexatriyn-1-yl, 1,4-hexadiyn-1-yl, 1,4-hexadiyn-3-yl, 1,5-hexadiyn-1-yl, 1,5-hexadiyn-3-yl, 2-hexyn-1-yl, 2,5-hexadiyn-1-yl, 3-hexyn-1-yl, 3-hexyn-2-yl, 3,5-hexadiyn-2-yl, 4-hexyn-1-yl, 4-hexyn-2-yl, 4-hexyn-3-yl, 5-hexyn-1-yl, 5-hexyn-2-yl, 5-hexyn-3-yl, 2-methyl-3-pentyn-1-yl, 2-methyl-3-pentyn-2-yl, 2-methyl-4-pentyn-1-yl, 2-methyl-4-pentyn-2-yl, 2-methyl-4-pentyn-3-yl, 3-methyl-1-pentyn-1-yl, 3-methyl-4-pentyn-1-yl, 3-methyl-4-pentyn-2-yl, 3-methyl-1,4-pentadiyn-1-yl, 3-methyl-1,4-pentadiyn-3-yl, 3-methyl-4-pentyn-1-yl, 3-methyl-4-pentyn-3-yl, 4-methyl-1-pentyn-1-yl and 4-methyl-2-pentyn-1-yl.

The term “C₄-cycloalkane,” as used herein, means cyclobutane.

The term “C₅-cycloalkane,” as used herein, means cyclopentane.

The term “C₆-cycloalkane,” as used herein, means cyclohexane.

The term “C₄-cycloalkene,” as used herein, means cyclobutene and 1,3-cyclobutadiene.

The term “C₅-cycloalkene,” as used herein, means cyclopentene and 1,3-cyclopentadiene.

The term “C₆-cycloalkene,” as used herein, means cyclohexene, 1,3-cyclohexadiene and 1,4-cyclohexadiene.

The term “C₃-cycloalkenyl,” as used herein, means cycloprop-1-en-1-yl and cycloprop-2-en-1-yl.

The term “C₄-cycloalkenyl,” as used herein, means cyclobut-1-en-1-yl and cyclobut-2-en-1-yl.

The term “C₅-cycloalkenyl,” as used herein, means cyclopent-1-en-1-yl, cyclopent-2-en-1-yl, cyclopent-3-en-1-yl and cyclopenta-1,3-dien-1-yl.

The term “C₆-cycloalkenyl,” as used herein, means cyclohex-1-en-1-yl, cyclohex-2-en-1-yl, cyclohex-3-en-1-yl, cyclohexa-1,3-dien-1-yl, cyclohexa-1,4-dien-1-yl, cyclohexa-1,5-dien-1-yl, cyclohexa-2,4-dien-1-yl and cyclohexa-2,5-dien-1-yl.

The term “C₃-cycloalkyl,” as used herein, means cycloprop-1-yl.

The term “C₄-cycloalkyl,” as used herein, means cyclobut-1-yl.

The term “C₅-cycloalkyl,” as used herein, means cyclopent-1-yl.

The term “C₆-cycloalkyl,” as used herein, means cyclohex-1-yl.

Compounds of this invention contain asymmetrically substituted carbon atoms in the R or S configuration, in which the terms “R” and “S” are as defined by the IUPAC 1974 Recommendations for Section E, Fundamental Stereochemistry, Pure Appl. Chem. (1976) 45, 13-10. Compounds having asymmetrically substituted carbon atoms with equal amounts of R and S configurations are racemic at those carbon atoms. Atoms with an excess of one configuration over the other are assigned the configuration present in the higher amount, preferably an excess of about 85%-90%, more preferably an excess of about 95%-99%, and still more preferably an excess greater than about 99%. Accordingly, this invention includes racemic mixtures, relative and absolute stereoisomers, and mixtures of relative and absolute stereoisomers.

Compounds of this invention may also contain carbon-carbon double bonds or carbon-nitrogen double bonds in the Z or E configuration, in which the term “Z” represents the larger two substituents on the same side of a carbon-carbon or carbon-nitrogen double bond and the term “E” represents the larger two substituents on opposite sides of a carbon-carbon or carbon-nitrogen double bond. The compounds may also exist as an equilibrium mixture of Z or E configurations.

Compounds of this invention containing NH, C(O)OH, OH or SH moieties may have attached thereto prodrug-forming moieties. The prodrug-forming moieties are removed by metabolic processes and release the compounds having the freed hydroxyl, amino or carboxylic acid in vivo. Prodrugs are useful for adjusting such pharmacokinetic properties of the compounds as solubility and/or hydrophobicity, absorption in the gastrointestinal tract, bioavailability, tissue penetration, and rate of clearance.

Compounds having formula (I) may exist as an acid addition salts, basic addition salts or zwitterions. Salts of the compounds are prepared during their isolation or following their purification. Acid addition salts of the compounds are those derived from the reaction of the compounds with an acid. For example, the acetate, adipate, alginate, bicarbonate, citrate, aspartate, benzoate, benzenesulfonate, bisulfate, butyrate, camphorate, camphorsufonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetic, trifluoroacetic, para-toluenesulfonate, and undecanoate salts of the compounds and prodrugs thereof are contemplated as being embraced by this invention. Basic addition salts of the compounds are those derived from the reaction of the compounds with the hydroxide, carbonate or bicarbonate of cations such as lithium, sodium, potassium, calcium, and magnesium.

The compounds having formula (I) may be administered, for example, bucally, ophthalmically, orally, osmotically, parenterally (intramuscularly, intraperintoneally intrasternally, intravenously, subcutaneously), rectally, topically, transdermally, or vaginally.

Therapeutically effective amounts of compounds having formula (I) depend on recipient of treatment, disorder being treated and severity thereof, composition containing it, time of administration, route of administration, duration of treatment, its potency, its rate of clearance and whether or not another drug is co-administered. The amount of a compound of this invention having formula (I) used to make a composition to be administered daily to a patient in a single dose or in divided doses is from about 0.03 to about 200 mg/kg body weight. Single dose compositions contain these amounts or a combination of submultiples thereof.

Compounds having formula (I) may be administered with or without an excipient. Excipients include, for example, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof.

Excipients for preparation of compositions comprising a compound having formula (I) to be administered orally in solid dosage form include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl celluose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof. Excipients for preparation of compositions comprising a compound of this invention having formula (I) to be administered ophthalmically or orally in liquid dosage forms include, for example, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water and mixtures thereof. Excipients for preparation of compositions comprising a compound of this invention having formula (I) to be administered osmotically include, for example, chlorofluorohydrocarbons, ethanol, water and mixtures thereof. Excipients for preparation of compositions comprising a compound of this invention having formula (I) to be administered parenterally include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof. Excipients for preparation of compositions comprising a compound of this invention having formula (I) to be administered rectally or vaginally include, for example, cocoa butter, polyethylene glycol, wax and mixtures thereof.

To determine antibacterial activity of compounds of this invention, twelve petri dishes, each containing successive aqueous dilutions of test compounds in sterilized Brain Heart Infusion agar (Difco 0418-01-5) (10 mL), were inoculated with 1:100 dilutions of Staphylococcus aureus 6538P using a Steers replicator block (or 1:10 dilutions for slow-growing Streptococcus strains), co-incubated at 35-37° C. for 20-24 hours with a plate having no compound, and inspected visually to provide the minimum inhibitory concentration (MIC), in μg/mL, by which is meant the lowest concentration of the test compound which yielded no growth, a slight haze, or sparsely isolated colonies on the inoculums spot as compared to growth in the control plate.

The MIC values of representative compounds tested against Staphylococcus aureus 6538P, 1, 1, 1, 2, 2, 2, 2, 2, 2, 2.5, 3, 3, 4, 4, 4, 4, 4, 4, 4, 4, 4, 4, 6, 8, 8, 8, 8, 8, 8, 16, 16, 16, 16, 16, 32, 64, 64, 128, 128, 128 and 128. These data demonstrate the antibacterial utility of the compounds having formula (I).

Compounds having formula (I) may be made by synthetic chemical processes, examples of which are shown below. It is meant to be understood that the order of the steps in the processes may be varied, reagents, solvents, and reaction conditions may be substituted for those specifically mentioned, and vulnerable moieties may be protected and deprotected, as necessary, by NH, C(O)OH, OH, SH protecting groups.

The term “NH protecting group,” as used herein, means trichloroethoxycarbonyl, tribromoethoxycarbonyl, benzyloxycarbonyl, para-nitrobenzylcarbonyl, ortho-bromobenzyloxycarbonyl, chloroacetyl, dichloroacetyl, trichloroacetyl, trifluoroacetyl, phenylacetyl, formyl, acetyl, benzoyl, tert-amyloxycarbonyl, tert-butoxycarbonyl, para-methoxybenzyloxycarbonyl, 3,4-dimethoxybenzyloxycarbonyl, 4-(phenylazo)benzyloxycarbonyl, 2-furfuryloxycarbonyl, diphenylmethoxycarbonyl, 1,1-dimethylpropoxy-carbonyl, isopropoxycarbonyl, phthaloyl, succinyl, alanyl, leucyl, 1-adamantyloxycarbonyl, 8-quinolyloxycarbonyl, benzyl, diphenylmethyl, triphenylmethyl, 2-nitrophenylthio, methanesulfonyl, para-toluenesulfonyl, N,N-dimethylaminomethylene, benzylidene, 2-hydroxybenzylidene, 2-hydroxy-5-chlorobenzylidene, 2-hydroxy-1-naphthyl-methylene, 3-hydroxy-4-pyridylmethylene, cyclohexylidene, 2-ethoxycarbonylcyclohexylidene, 2-ethoxycarbonylcyclopenty-lidene, 2-acetylcyclohexylidene, 3,3-dimethyl-5-oxycyclohexylidene, diphenylphosphoryl, dibenzylphosphoryl, 5-methyl-2-oxo-2H-1,3-dioxol-4-yl-methyl, trimethylsilyl, triethylsilyl, and triphenylsilyl.

The term “C(O)OH protecting group,” as used herein, means methyl, ethyl, n-propyl, isopropyl, 1,1-dimethylpropyl, n-butyl, tert-butyl, phenyl, naphthyl, benzyl, diphenylmethyl, triphenylmethyl, para-nitrobenzyl, para-methoxybenzyl, bis(para-methoxyphenyl)methyl, acetylmethyl, benzoylmethyl, para-nitrobenzoylmethyl, para-bromobenzoylmethyl, para-methanesulfonylbenzoylmethyl, 2-tetrahydropyranyl 2-tetrahydrofuranyl, 2,2,2-trichloro-ethyl, 2-(trimethylsilyl)ethyl, acetoxymethyl, propionyloxymethyl, pivaloyloxymethyl, phthalimidomethyl, succinimidomethyl, cyclopropyl, cyclobutyl, cyclopentyl, cyclohexyl, methoxymethyl, methoxyethoxymethyl, 2-(trimethylsilyl)ethoxymethyl, benzyloxymethyl, methylthiomethyl, 2-methylthioethyl, phenylthiomethyl, 1,1-dimethyl-2-propenyl, 3-methyl-3-butenyl, allyl, trimethylsilyl, triethylsilyl, triisopropylsilyl, diethylisopropylsilyl, tert-butyldimethylsilyl, tert-butyldiphenylsilyl, diphenylmethylsilyl, and tert-butylmethoxyphenylsilyl.

The term “OH or SH protecting group,” as used herein, means benzyloxycarbonyl, 4-nitrobenzyloxycarbonyl, 4-bromobenzyloxycarbonyl, 4-methoxybenzyloxycarbonyl, 3,4-dimethoxybenzyloxycarbonyl, methoxycarbonyl, ethoxycarbonyl, tert-butoxycarbonyl, 1,1-dimethylpropoxycarbonyl, isopropoxycarbonyl, isobutyloxycarbonyl, diphenylmethoxycarbonyl, 2,2,2-trichloroethoxycarbonyl, 2,2,2-tribromoethoxycarbonyl, 2-(trimethylsilyl)ethoxycarbonyl, 2-(phenylsulfonyl)ethoxycarbonyl, 2-(triphenylphosphonio)ethoxycarbonyl, 2-furfuryloxycarbonyl, 1-adamantyloxycarbonyl, vinyloxycarbonyl, allyloxycarbonyl, S-benzylthiocarbonyl, 4-ethoxy-1-naphthyloxycarbonyl, 8-quinolyloxycarbonyl, acetyl, formyl, chloroacetyl, dichloroacetyl, trichloroacetyl, trifluoroacetyl, methoxyacetyl, phenoxyacetyl, pivaloyl, benzoyl, methyl, tert-butyl, 2,2,2-trichloroethyl, 2-trimethylsilylethyl, 1,1-dimethyl-2-propenyl, 3-methyl-3-butenyl, allyl, benzyl (phenylmethyl), para-methoxybenzyl, 3,4-dimethoxybenzyl, diphenylmethyl, triphenylmethyl, tetrahydrofuryl, tetrahydropyranyl, tetrahydrothiopyranyl, methoxymethyl, methylthiomethyl, benzyloxymethyl, 2-methoxyethoxymethyl, 2,2,2-trichloro-ethoxymethyl, 2-(trimethylsilyl)ethoxymethyl, 1-ethoxyethyl, methanesulfonyl, para-toluenesulfonyl, trimethylsilyl, triethylsilyl, triisopropylsilyl, diethylisopropylsilyl, tert-butyldimethylsilyl, tert-butyldiphenylsilyl, diphenylmethylsilyl, and tert-butylmethoxyphenylsilyl.

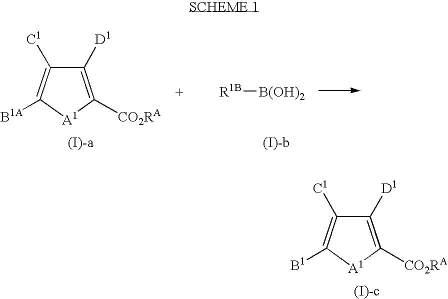

Compounds having formula (I)-c may be prepared by reacting compounds having formula (I)-a, wherein R^{A} is a C(O)OH protecting group such as C₁-alkyl or C₂-alkyl, formula (I)-b, tetrakis(triphenylphosphine)palladium(0) and a base. The moiety represented by R^{1B} may be unsubstituted or substituted phenyl or unsubstituted or substituted cyclohexen-1-yl. If cyclohexen-1-yl boronic acid is used, reduction of the product with hydrogen and a catalyst such as palladium or platinum may be used to provide unsubstituted or substituted cyclohexyl at that position. Examples of bases include triethylamine, diisopropylethylamine, cesium carbonate or cesium fluoride. The reaction is typically conducted in solvents auch as tetfahydrofuran or 1,2-dimethozyethane at temperatures between about 50° C. and 110° C.

Specific examples are described below.

### EXAMPLE 1

1M phenylmagnesium bromide in THF (11.2 mL) at −78° C. was treated with 3-phenylpropionaldehyde (1 g) in THF (50 mL), stirred for 6 hours, warmed to room temperature, and treated with saturated ammonium chloride. The extract was washed with water and brine and dried (MgSO₄), filtered and concentrated. The concentrate was flash chromatographed with 10% ethyl acetate/hexanes.

### EXAMPLE 2

This compound was prepared by substituting phenyl magnesium bromide with 2-lithiothiophene in EXAMPLE 1.

### EXAMPLE 3

This compound was prepared by substituting phenylpropionaldehyde with 3-cyclohexylpropionaldehyde in EXAMPLE 1.

### EXAMPLE 4

This compound was prepared by substituting phenyl magnesium bromide with 2-lithiothiophene in EXAMPLE 3.

### EXAMPLE 5

This compound was prepared by substituting phenyl magnesium bromide with 4-biphenylmagnesium bromide in EXAMPLE 3.

### EXAMPLE 6

This compound was prepared by substituting phenyl magnesium bromide with tolylmagnesium bromide in EXAMPLE 3.

### EXAMPLE 7

This compound was prepared by substituting phenyl magnesium bromide with 2-naphthylmagnesium bromide in EXAMPLE 3.

### EXAMPLE 8

3-cyclohexylpropionaldehyde (1 g) in dichloromethane (30 mL) at 25° C. was treated with 2-trimethylsilylthiazole (1.2 g), stirred for 6 hours, treated with THF (30 mL) and 1M TBAF in THF (8 mL), stirred for 2 hours, and concentrated. The concentrate in ethyl was washed with saturated NaHCO₃, water, and brine, and dried (MgSO₄), filtered, and concentrated. The concentrate was flash chromatographed with 5-20% ethyl acetate/hexanes.

### EXAMPLE 9

A mixture of ethyl 3-methyl-4-bromobenzoate (32 g) in diethyl ether (450 mL) at −78° C. was treated with 1M lithium aluminum hydride in THF (190 mL), stirred for 1.5 hours, treated with brine, and filtered. The filtrate was washed with 1M HCl and brine, and dried (Na₂SO₄), filtered, and concentrated.

### EXAMPLE 10

A mixture of EXAMPLE 9 (25 g) in ethanol (300 mL) was treated with concentrated sulfuric acid (12 mL), stirred at reflux for 3 days, concentrated to 50 mL, treated with sodium carbonate to pH 8, and extracted with ethyl acetete. The extract was dried (Na₂SO₄), filtered, and concentrated.

### EXAMPLE 11

A mixture of EXAMPLE 10 (23 g), bis(pinicolato)diboron (32 g), potassium carbonate (40 g) in 1,4-dioxane was treated with [1,1′-bis(diphenylphosphine)ferrocene]dichloropalladium dichloromethane (4.67 g), stirred for 6 hours at 90° C., and concentrated to 80 mL. The concentrate was treated with ethyl acetate (500 mL), washed with water, 1M HCl, and brine, and dried (Na₂SO₄), filtered, and concentrated. The concentrate was flash chromatographed on silica gel with 10-30% ethyl acetate/hexanes.

### EXAMPLE 12

An alcohol (one of EXAMPLES 1-8) (0.321 mmol) in tert-butanol (0.2 mL) at 25° C. was treated with 1M potassium tert-butoxide in THF (0.32 mL), stirred for 1 hour, treated with EXAMPLE (75 mg) in tert-butanol (5 mL) and of potassium iodide (105 mg), stirred at 50° C. for 1 hour, and concentrated. A mixture of the concentrate and lithium hydroxide monohydrate (45 mg) in 3:2 THF/water (2 mL) at 25° C. was stirred for 72 hours, treated with saturated aqueous NH₄Cl and acetic acid and extracted with dichloromethane. The extract was concentrated, and the concentrate was purified by reverse phase HPLC.

The foregoing is merely illustrative of the invention and is not intended to limit the same to the disclosed compounds and processes. Variations and changes which are obvious to one skilled in the art are intended to be within the scope and nature of the invention as defined in the claims.

What is claimed is:

1. A compound having formula (I) 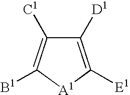
wherein
A is O or S;
one of B¹ or C¹ is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;
D¹ is H, OH, OCH₃, F, Cl or Br; and
E¹ is CO₂H or tetrazolyl; or
A¹ is C(R²)═C(R³);
one of B¹, C¹ or R² is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;
R³ and D¹ are independently H, OH, OCH₃, F, Cl or Br;
E¹ is CO₂H or tetrazolyl; or
A¹ is C(H)═C(H);
D¹ and E¹ are together and, with the atoms to which each is attached, are furan-2-carboxylic acid or furan-2-tetrazolyl; and
one of B¹ or C¹ is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;
R¹ is C₁-alkyl, C₂-alkyl, C₃-alkyl, C₄-alkyl, C₅-alkyl, C₆-alkyl, C₃-alkenyl, C₄-alkenyl, C₅-alkenyl, C₆-alkenyl, C₃-alkynyl, C₄-alkynyl, C₅-alkynyl or C₆-alkynyl;
R⁴ is R⁵, R⁶, R⁷ or R⁸;
R⁵ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;
R⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two or three independently selected R⁹, OR⁹, SR⁹, S(O)R⁹, SO₂R⁹, NHR⁹, N(R⁹)₂, C(O)R⁹, C(O)NH₂, C(O)NHR⁹, NHC(O)R⁹, NHSO₂R⁹, NHC(O)OR⁹, SO₂NH₂, SO₂NHR⁹, SO₂N(R⁹)₂ or NHC(O)NHR⁹, substituents;
R⁹ is R¹⁰, R¹¹, R¹² or R¹³;
R¹⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R¹¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R¹² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;
R¹³ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two independently selected R¹⁴ substituents;
R¹⁴ is R¹⁵, R¹⁶, R¹⁷ or R¹⁸;
R¹⁵ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R¹⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R¹⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;
R¹⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two or three independently selected R¹⁹ substituents;
R¹⁹ is R²⁰, R²¹ or R²²;
R²⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R²¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R²² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;
wherein each foregoing cyclic moiety is independently unsubstituted, further unsubstituted, substituted or further substituted with one or two or three independently selected R²³, OR²³, SR²³, S(O)R²³, SO₂R²³, C(O)R²³, CO(O)R²³, OC(O)R²³, OC(O)OR²³, NH₂, NHR²³, N(R²³)₂, NHC(O)R²³, NR²³C(O)R²³C(O)NH₂, C(O)NHR²³, C(O)N(R²³)₂, SO₂NH₂, SO₂NHR²³, SO₂N(R²³)₂, CF₃, CF₂CF₃, C(O)H, OH, NO₂, CF₃, CF₂CF₃, OCF₃, OCF₂CF₃, F, Cl, Br or I substituents;
R²³ is R²⁴, R²⁵, R²⁶ or R²⁷;
R²⁴ is phenyl which is unfused or fused with benzene;
R²⁵ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl;
R²⁶ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N; and
R²⁷ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two independently selected phenyl, furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl 1,2,3-triazolyl, CN, NH₂, NH(CH₃), N(CH₃)₂, CF₃, OCF₃, CF₂CF₃, OCF₂CF₃, F, Cl, Br or I substituents;
wherein the moieties represented by R²⁴, R²⁵ and R²⁶ are unsubstituted or substituted with one or two or three independently selected C₁-alkyl, C₂-alkyl, C₃-alkyl, C₄-alkyl, C₅-alkyl, C₆-alkyl, CN, NH₂, NH(CH₃), N(CH₃)₂, CF₃, OCF₃, CF₂CF₃, OCF₂CF₃, F, Cl, Br or I substituents,
or a therapeutically acceptable salt thereof.

2. The compound of claim 1 having formula (I) wherein
A is O or S;
one of B¹ or C¹ is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;
D¹ is H, OH, OCH₃, F, Cl or Br; and
E¹ is CO₂H or tetrazolyl; or
A¹ is C(R²)═C(R³);
one of B¹, C¹ or R² is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;
R³ and D¹ are independently H, OH, OCH₃, F, Cl or Br;
E¹ is CO₂H or tetrazolyl; or
A¹ is C(H)═C(H);
D¹ and E¹ are together and, with the atoms to which each is attached, are furan-2-carboxylic acid or furan-2-tetrazolyl; and
one of B¹ or C¹ is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;
R¹ is C₁-alkyl, C₂-alkyl, C₃-alkyl, C₄-alkyl, C₅-alkyl, C₆-alkyl, C₃-alkenyl, C₄-alkenyl, C₅-alkenyl, C₆-alkenyl, C₃-alkynyl, C₄-alkynyl, C₅-alkynyl or C₆-alkynyl;
R⁴ is R⁵, R⁶, R⁷ or R⁸;
R⁵ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene;
R⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;
R⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two or three independently selected R⁹, OR⁹, SR⁹, S(O)R⁹, SO₂R⁹, NHR⁹, N(R⁹)₂, C(O)R⁹, C(O)NH₂, C(O)NHR⁹, NHC(O)R⁹, NHSO₂R⁹, NHC(O)OR⁹, SO₂NH₂, SO₂NHR⁹, SO₂N(R⁹)₂ or NHC(O)NHR⁹, substituents;
R⁹ is R¹⁰, R¹¹, R¹² or R¹³;
R¹⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R¹¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene;
R¹² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;
R¹³ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two independently selected R¹⁴ substituents;
R¹⁴ is R¹⁵, R¹⁶, R¹⁷ or R¹⁸;
R¹⁵ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R¹⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene;
R¹⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;
R¹⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two or three independently selected R¹⁹ substituents;
R¹⁹ is R²⁰, R²¹ or R²²;
R²⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R²¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene;
R²² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;
wherein each foregoing cyclic moiety is independently unsubstituted, further unsubstituted, substituted or further substituted with one or two or three independently selected R²³, NH₂, NHR²³, NHC(O)R²³, OH, NO₂, CF₃, CF₂CF₃, OCF₃, OCF₂CF₃, F, Cl, Br or I substituents;
R²³ is R²⁴, R²⁵, R²⁶ or R²⁷;
R²⁴ is phenyl which is unfused or fused with benzene;
R²⁵ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl;
R²⁶ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH; and
R² is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two independently selected phenyl, furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl 1,2,3-triazolyl, CN, NH₂, NH(CH₃), N(CH₃)₂, CF₃, OCF₃, CF₂CF₃, OCF₂CF₃, F, Cl, Br or I substituents;
wherein the moieties represented by R²⁴, R²⁵ and R²⁶ are unsubstituted or substituted with one or two or three independently selected C₁-alkyl, CN, NH₂, NH(CH₃), N(CH₃)₂, CF₃, OCF₃, CF₂CF₃, OCF₂CF₃, F, Cl, Br or I substituents,
or a therapeutically acceptable salt thereof.

3. The compound of claim 2 having formula (I), wherein
A¹ is O or S;
one of B¹ or C¹ is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;
D¹ is H, OH, OCH₃, F, Cl or Br; and
E¹ is CO₂H or tetrazolyl; or
A¹ is C(R²)═C(R³);
one of B¹, C¹ or R² is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;
R³ and D¹ are independently H, OH, OCH₃, F, Cl or Br;
E¹ is CO₂H or tetrazolyl; or
A¹ is C(H)═C(H);
D¹ and E¹ are together and, with the atoms to which each is attached, are furan-2-carboxylic acid or furan-2-tetrazolyl; and
one of B¹ or C¹ is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;
R¹ is C₁-alkyl, C₂-alkyl, C₃-alkyl, C₄-alkyl, C₅-alkyl, C₆-alkyl, C₃-alkenyl, C₄-alkenyl, C₅-alkenyl, C₆-alkenyl, C₃-alkynyl, C₄-alkynyl, C₅-alkynyl or C₆-alkynyl;
R⁴ is R⁵, R⁶, R⁷ or R⁸;
R⁵ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene;
R⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;
R⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two or three independently selected R⁹, OR⁹, SR⁹, S(O)R⁹, SO₂R⁹, NHR⁹, N(R⁹)₂, C(O)R⁹, C(O)NH₂, C(O)NHR⁹, NHC(O)R⁹, NHSO₂R⁹, NHC(O)OR⁹, SO₂NH₂, SO₂NHR⁹, SO₂N(R⁹)₂ or NHC(O)NHR⁹, substituents;
R⁹ is R¹⁰, R¹¹, R¹² or R¹³;
R¹⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R¹¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene;
R¹² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;
R¹³ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two independently selected R¹⁴ substituents;
R¹⁴ is R¹⁵, R¹⁶, R¹⁷ or R¹⁸;
R¹⁵ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R¹⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene;
R¹⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;
R¹⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two or three independently selected R¹⁹ substituents;
R¹⁹ is R²⁰, R²¹ or R²²;
R²⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R²¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene;
R²² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;
wherein each foregoing cyclic moiety is independently unsubstituted, further unsubstituted, substituted or further substituted with one or two or three independently selected R²³, NH₂, NHR²³, NHC(O)R²³, OH, NO₂, CF₃, CF₂CF₃, OCF₃, OCF₂CF₃, F, Cl, Br or I substituents;
R²³ is R²⁴, R²⁵, R²⁶ or R²⁷;
R²⁴ is phenyl which is unfused or fused with benzene;
R²⁵ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl;
R²⁶ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH; and
R²⁷ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two independently selected phenyl, furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl 1,2,3-triazolyl, CN, NH₂, NH(CH₃), N(CH₃)₂, CF₃, OCF₃, CF₂CF₃, OCF₂CF₃, F, Cl, Br or I substituents;
wherein the moieties represented by R²⁴, R²⁵ and R²⁶ are unsubstituted or substituted with one or two or three independently selected C₁-alkyl, CN, NH₂, NH(CH₃), N(CH₃)₂, CF₃, OCF₃, CF₂CF₃, OCF₂CF₃, F, Cl, Br or I substituents,
or a therapeutically acceptable salt thereof.

4. The compound of claim 3 having formula (I) wherein
A¹ is O or S;
one of B¹ or C¹ is H, OH, OR¹, CN, F, Cl or Br, and the other is phenyl or C₆-cycloalkyl, each of which is substituted with R⁴, OR⁴, SR⁴, S(O)R⁴, SO₂R⁴, C(O)NHR⁴, C(O)N(R⁴)₂, NHC(O)R⁴, SO₂NHR⁴ or NHSO₂R⁴;
D¹ is H; and
E¹ is CO₂H; or
A¹ is C(R²)═C(R³);
one of B¹, C¹ or R² is H, OH, OR¹, CN, F, Cl or Br, and the other is phenyl or C₆-cycloalkyl, each of which is substituted with R⁴, OR⁴, SR⁴, S(O)R⁴, SO₂R⁴, C(O)NHR⁴, NHC(O)R⁴, SO₂NHR⁴ or NHSO₂R⁴;
R³ and D¹ are independently H, OH, OCH₃, F, Cl or Br;
E¹ is CO₂H or tetrazolyl; or
A¹ is C(H)═C(H);
D¹ and E¹ are together and, with the atoms to which each is attached, are furan-2-carboxylic acid; and
one of B¹ or C¹ is H, OH, OR¹, CN, F, Cl or Br, and the other is phenyl or C₆-cycloalkyl, each of which is substituted with R⁴, OR⁴, SR⁴, S(O)R⁴, SO₂R⁴, C(O)NHR⁴, NHC(O)R⁴, SO₂NHR⁴ or NHSO₂R⁴;
R¹ is C₁-alkyl, C₂-alkyl, C₃-alkyl, C₄-alkyl, C₅-alkyl or C₆-alkyl;
R⁴ is R⁵, R⁶, R⁷ or R⁸;
R⁵ is phenyl which is unfused or fused with benzene;
R⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl;
R⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;
R⁸ is C₁-alkyl, C₂-alkyl, C₃-alkyl, C₄-alkyl, C₅-alkyl or C₆-alkyl, each of which is substituted with one or two or three independently selected R⁹, OR⁹, SR⁹, S(O)R⁹, SO₂R⁹, NHR⁹, C(O)R⁹, C(O)NHR⁹, NHC(O)R⁹, NHSO₂R⁹, NHC(O)OR⁹, SO₂NH₂, SO₂NHR⁹ or NHC(O)NHR⁹ substituents;
R⁹ is R¹⁰, R¹¹, R¹² or R¹³;
R¹⁰ is phenyl which is unfused or fused with benzene;
R¹¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl;
R¹² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;
R¹³ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two independently selected R¹⁴ substituents;
R¹⁴ is R¹⁵, R¹⁶, R¹⁷ or R¹⁸;
R¹⁵ is phenyl which is unfused or fused with benzene;
R¹⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl;
R¹⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;
R¹⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two or three independently selected R¹⁹ substituents;
R¹⁹ is R²⁰, R²¹ or R²²;
R²⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R²¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl;
R²² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH;
wherein the phenyl and C₆-cycloalkyl moieties represented by B¹, C¹ or R² are further unsubstituted or substituted with R²³, NH₂, NHC(O)R²³, OH, NO₂ or Cl, wherein R²³ is morpholinyl or phenyl substituted with C₁-alkyl; and
the moieties represented by R¹⁵, R¹⁶ and R¹⁷ are unsubstituted or substituted with one or two independently selected C₁-alkyl, phenyl or OH substituents,
or a therapeutically acceptable salt thereof.

5. The compound of claim 4 having formula (I) which is
3-chloro-4′-((cyclohex-3-yl-1-(1,3-thiazol-5-yl)propoxy)methyl)-6-fluoro-2′-methyl(1,1′-biphenyl)-4-carboxylic acid,
5-(4-((cyclohex-3-yl-1-(1,3-thiazol-5-yl)propoxy)methyl)-2-methylphenyl)-2-thiophenecarboxylic acid,
3′-chloro-4′-((cyclohex-3-yl-1-(1,3-thiazol-5-yl)propoxy)methyl)-4-methoxy-6′-methyl(1,1′-biphenyl)-3-carboxylic acid,
5-(4-((cyclohex-3-ylpropoxy)methyl)-2-methylphenyl)-2-thiophenecarboxylic acid,
2′-(2-methylphenyl)-4′-((cyclohex-3-yl-1-(1,3-thiazol-5-yl)propoxy)methyl)(1,1′-biphenyl)-4-carboxylic acid,
2′-(2-methylphenyl)-4′-((cyclohex-3-yl-1-(1,3-thiazol-5-yl)propoxy)methyl)(1,1′-biphenyl)-3-carboxylic acid,
2′-(2-methylphenyl)-4′-(((3-methylphenylmethyl)thio)methyl)(1,1′-biphenyl)-3-carboxylic acid,
4′-((cyclohex-3-yl-1-(1,3-thiazol-5-yl)propoxy)methyl)-2′-methyl(1,1′-biphenyl)-3-carboxylic acid,
4-(4-propylcyclohexyl)benzoic acid,
4′-((cyclohex-3-yl-2-(1,3-thiazol-5-yl)propoxy)methyl)(1,1′-biphenyl)-3-carboxylic acid,
2′-methyl-4′-(((3-methylphenylmethyl)thio)methyl)(1,1′-biphenyl)-4-carboxylic acid,
2′-chloro-4′-(((3-methylphenylmethyl)thio)methyl)(1,1′-biphenyl)-4-carboxylic acid,
2′-methyl-4′-((pentyloxy)methyl)(1,1′-biphenyl)-4-carboxylic acid,
4′-((3-cyclohexyl-1-(furan-2-yl)propoxy)methyl)-2′-methyl(1,1′-biphenyl)-4-carboxylic acid,
4′-((cyclohex-3-yl-1-(thien-2-yl)propoxy)methyl)-2′-methyl(1,1′-biphenyl)-4-carboxylic acid,
3′-chloro-4′-((cyclohex-3-yl-1-(1,3-thiazol-5-yl)propoxy)methyl)(1,1′-biphenyl)-4-carboxylic acid,
4′-((cyclohex-3-l-1-(1,3-thiazol-5-yl)propoxy)methyl)-2,2′-dimethyl(1,1′-biphenyl)-4-carboxylic acid,
5-(4-((cyclohex-3-ylpropoxy)methyl)-2-methylphenyl)-1-benzofuran-2-carboxylic acid,
4′-((cyclohex-3-yl-1-(1,3-thiazol-5-yl)propoxy)methyl)-3-fluoro-2′-methyl(1,1′-biphenyl)-4-carboxylic acid,
4′-((cyclohex-3-yl-1-(1,3-thiazol-5-yl)propoxy)methyl)-2-methoxy-2′-methyl(1,1′-biphenyl)-4-carboxylic acid,
5-(4′-((cyclohex-3-yl-1-(1,3-thiazol-5-yl)propoxy)methyl)-2′-methyl(1,1′-biphenyl)-4-yl)-1H-tetraazole,
4′-((cyclohex-3-yl-1-(1,3-thiazol-5-yl)propoxy)methyl)-3-hydroxy(1,1′-biphenyl)-4-carboxylic acid,
4-(4-butylcyclohexyl)benzoic acid,
2′-chloro-4′-((cyclohex-3-yl-1-(1,3-thiazol-5-yl)propoxy)methyl)(1,1′-biphenyl)-3-carboxylic acid,
4′-((cyclohex-3-ylpropoxy)methyl)-2′-methyl(1,1′-biphenyl)-3-carboxylic acid,
6-(4-((cyclohex-3-yl-1-(1,3-thiazol-5-yl)propoxy)methyl)-2-methylphenyl)-1-benzofuran-2-carboxylic acid,
5-(4′-((cyclohex-3-yl-1-(1,3-thiazol-5-yl)propoxy)methyl)-4-fluoro-2′-methyl(1,1′-biphenyl)-3-yl)-1H-tetraazole,
5-(4′-((cyclohex-3-yl-1-(1,3-thiazol-5-yl)propoxy)methyl)-3-fluoro-2′-methyl(1,1′-biphenyl)-4-yl)-1H-tetraazole,
4′-((cyclohex-3-yl-1-(1,3-thiazol-5-yl)propoxy)methyl)-2′-methyl(1,1′-biphenyl)-4-carboxylic acid,
4′-((cyclohex-3-yl-1-(1,3-thiazol-5-yl)propoxy)methyl)-4-hydroxy-2′-methyl(1,1′-biphenyl)-3-carboxylic acid,
5-(4′-((cyclohex-3-ylpropoxy)methyl)-2′-nitro(1,1′-biphenyl)-3-yl)-2H-tetraazole,
2′-amino-4′-((cyclohex-3-yl-1-(1,3-thiazol-5-yl)propoxy)methyl)(1,1′-biphenyl)-3-carboxylic acid,
4′-((cyclohex-3-yl-1-(1,3-thiazol-5-yl)propoxy)methyl)-2′-((2-morpholinylcarbonyl)amino)(1,1′-biphenyl)-3-carboxylic acid,
2-fluoro-4-(4-pentylcyclohexyl)benzoic acid,
2′-(3-aminopropyl)-4′-((cyclohex-3-yl-1-(1,3-thiazol-5-yl)propoxy)methyl)-3-fluoro(1,1′-biphenyl)-4-carboxylic acid,
3-fluoro-2′-methyl-4′-(((1-pentylpropyn-2-yl)oxy)methyl)(1,1′-biphenyl)-4-carboxylic acid,
4′-((cyclohex-3-yl-1-(naphth-2-yl)propoxy)methyl)-3-fluoro-2′-methyl(1,1′-biphenyl)-4-carboxylic acid,
3-fluoro-2′-methyl-4′-(((1-pentylpropyn-2-yl)oxy)methyl)(1,1′-biphenyl)-4-carboxylic acid,
3-fluoro-4′-(((isoprop-1-yl-3-phenylpropyn-2-yl)oxy)methyl)-2′-methyl(1,1′-biphenyl)-4-carboxylic acid,
3-fluoro-2′-methyl-4′-(((pent-1-yl-3-(thien-2-l)propyn-2-yl)oxy)methyl)(1,1′-biphenyl)-4-carboxylic acid,
3-fluoro-2′-methyl-4′-((1-methyl-2,2-diphenylethoxy)methyl)(1,1′-biphenyl)-4-carboxylic acid,
3-fluoro-4′-((1-(5-hydroxy-2-phenyl-1,3-oxazol-4-yl)ethoxy)methyl)-2′-methyl(1,1′-biphenyl)-4-carboxylic acid,
4′-((1,3-diphenylpropoxy)methyl)-3-fluoro-2′-methyl(1,1′-biphenyl)-4-carboxylic acid,
4′-((cyclohex-3-yl-1-(thien-2-yl)propoxy)methyl)-3-fluoro-2′-methyl(1,1′-biphenyl)-4-carboxylic acid,
4′-((1-(1,1′-biphenyl)-4-ylcyclohex-3-yl propoxy)methyl)-3-fluoro-2′-methyl(1,1′-biphenyl)-4-carboxylic acid,
3-fluoro-2′-methyl-4′-((3-phenyl-1-(thien-2-yl)propoxy)methyl)(1,1′-biphenyl)-4-carboxylic acid,
4′-((cyclohex-3-yl-1-(1,3-thiazol-2-yl)propoxy)methyl)-3-fluoro-2′-methyl(1,1′-biphenyl)-4-carboxylic acid,
4′-((cyclohex-3-yl-1-phenylpropoxy)methyl)-3-fluoro-2′-methyl(1,1′-biphenyl)-4-carboxylic acid and 4′-((cyclohex-3-yl-1-(4-methylphenyl)propoxy)methyl)-3-fluoro-2′-methyl(1,1′-biphenyl)-4-carboxylic acid,
or a therapeutically acceptable salt thereof.

6. A composition for treating bacterial infection in a fish or a mammal, said composition comprising an excipient and a compound having formula (I), or therepeutically acceptable salt thereof.

7. A method of treating bacterial infection in a fish or a mammal, said method comprising administering to the fish or the mammal a compound having formula (I), 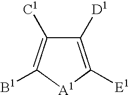
wherein
A¹ is O or S;
one of B¹ or C¹ is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;
D¹ is H, OH, OCH₃, F, Cl or Br; and
E¹ is CO₂H or tetrazolyl; or
A¹ is C(R²)═C(R³);
one of B¹, c or R¹ is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;
R³ and D¹ are independently H, OH, OCH₃, F, Cl or Br;
E¹ is CO₂H or tetrazolyl; or
A¹ is C(H)═C(H);
D¹ and E¹ are together and, with the atoms to which each is attached, are furan-2-carboxylic acid or furan-2-tetrazolyl; and
one of B¹ or C¹ is H, OH, OR¹, CN, F, Cl or Br, and the other is phenyl or C₆-cycloalkyl, each of which is substituted with one or two independently selected R⁴, OR⁴, SR⁴, S(O)R⁴, SO₂R⁴, C(O)NHR⁴, C(O)N(R⁴)₂, NHC(O)R⁴, NR⁴C(O)R⁴, SO₂NHR⁴, SO₂N(R⁴)₂, NHSO₂R⁴ or NR⁴SO₂R⁴ substituents;
R¹ is C₁-alkyl, C₂-alkyl, C₃-alkyl, C₄-alkyl, C₅-alkyl, C₆-alkyl, C₃-alkenyl, C₄-alkenyl, C₅-alkenyl, C₆-alkenyl, C₃-alkynyl, C₄-alkynyl, C₅-alkynyl or C₆-alkynyl;
R⁴ is R⁵, R⁶, R⁷ or R⁸;
R⁵ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R⁷ is C-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;
R⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two or three independently selected R⁹, OR⁹, SR⁹, S(O)R⁹, SO₂R⁹, NHR⁹, N(R⁹)₂, C(O)R⁹, C(O)NH₂, C(O)NHR⁹, NHC(O)R⁹, NHSO₂R⁹, NHC(O)OR⁹, SO₂NH₂, SO₂NHR⁹, SO₂N(R⁹)₂ or NHC(O)NHR⁹, substituents;
R⁹ is R¹⁰, R¹¹, R¹² or R¹³;
R¹⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R¹¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R¹² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;
R¹³ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two independently selected R¹⁴ substituents;
R¹⁴ is R¹⁵, R¹⁶, R¹⁷ or R¹⁸;
R¹⁵ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R¹⁶ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R¹⁷ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected 0, C(O), S, S(O), SO₂ or NH and one or two CH moieties unreplaced or replaced with N;
R¹⁸ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two or three independently selected R¹⁹ substituents;
R¹⁹ is R²⁰, R²¹ or R²²;
R²⁰ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R²¹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R²² is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;
wherein each foregoing cyclic moiety is independently unsubstituted, further unsubstituted, substituted or further substituted with one or two or three independently selected R²³, OR²³, SR²³, S(O)R²³, SO₂R²³, C(O)R²³, CO(O)R²³, OC(O)R²³, OC(O)OR²³, NH₂, NHR²³, N(R²³)₂, NHC(O)R²³, NR²³C(O)R²³, C(O)NH₂, C(O)NHR²³, C(O)N(R²³)₂, SO₂NH₂, SO₂NHR²³, SO₂N(R²³)₂, CF₃, CF₂CF₃, C(O)H, OH, NO₂, CF₃, CF₂CF₃, OCF₃, OCF₂CF₃, F, Cl, Br or I substituents;
R²³ is R²⁴, R²⁵, R²⁶ or R²⁷;
R²⁴ is phenyl which is unfused or fused with benzene;
R²⁵ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl or 1,2,3-triazolyl;
R²⁶ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N; and
R²⁷ is C₁-alkyl, C₂-alkyl, C₃-alkyl, C₄-alkyl, C₅alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl, each of which is unsubstituted or substituted with one or two independently selected phenyl, furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, thiazolyl, thienyl, triazinyl 1,2,3-triazolyl, CN, NH₂, NH(CH₃), N(CH₃)₂, CF₃, OCF₃, CF₂CF₃, OCF₂CF₃, F, Cl, Br or I substituents;
wherein the moieties represented by R²⁴, R²⁵ and R²⁶ are unsubstituted or substituted with one or two or three independently selected C₁-alkyl, C₂-alkyl, C₃-alkyl, C₄-alkyl, C₅-alkyl, C₆-alkyl, CN, NH₂, NH(CH₃), N(CH₃)₂, CF₃, OCF₃, CF₂CF₃, OCF₂CF₃, F, Cl, Br or I substituents,
or a therapeutically acceptable salt thereof.
